SEQUENCE LISTING (3 sequences)

SEQ ID NO: 1 - DNA, 10 bp, Artificial Sequence
  cis element decoy oligonucleotide
gggatttccc

SEQ ID NO: 2 - DNA, 20 bp, Artificial Sequence
  cis element decoy oligonucleotide
ccttgaagggatttccctcc

SEQ ID NO: 3 - DNA, 20 bp, Artificial Sequence
  Scramble decoy
ttgccgtacctgacttagcc

(19) United States
(12) Patent Application Publication
(10) Pub. No.: US 2006/0233815 A1
(43) Pub. Date: Oct. 19, 2006

(54) Agents for protection from neointimal formation in grafts comprising an nfkappab decoy

(75) Inventor: Yoshiki Sawa, Nishinomiya-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AnGes MG. Inc, Ibaraki-shi (JP)

(21) Appl. No.: 10/528,346

(22) PCT Filed: Dec. 27, 2002
(86) PCT No.: PCT/JP02/13805

§ 371 Date: Dec. 23, 2005

(30) Foreign Application Priority Data
Sep. 20, 2002 (JP) 2002-275884

(51) Int. Cl. A61K 39/00

(52) U.S. Cl. 424/184.1

(57) ABSTRACT

The present invention provides methods for using NFκB decoys to regulate (suppress) transcription activated by NFκB, and to suppress neointimal formation in grafts. Furthermore, the present invention relates to agents for protection from intimal thickening in vascular grafts that comprise NFκB decoys.

### TECHNICAL FIELD

The present invention relates to methods for regulating transcription activated by transcription factor, NFκB, in parts of blood vessels or vascular grafts. Specifically, the present invention relates to methods for suppressing neointimal formation in grafts, by introducing NFκB decoys into blood vessels or vascular grafts using a pressure-mediated method to regulate NFκB-activated transcription in vein grafts. Furthermore, the present invention relates to agents for protection from intimal thickening in vascular grafts comprising an NFκB decoy.

### BACKGROUND ART

Coronary artery reconstructions, and reconstructions of popliteal arteries below the knee and tibial arteries, are conventional methods of treatment for ischemic diseases. In such reconstructions, autologous internal thoracic arteries and great saphenous veins are commonly used. However, vascular occlusion is often known to occur, due to vascular thickening that results from vascular smooth muscle cell proliferation and such after angioplasty, artery bypass graft surgery, and organ transplant. Specifically, saphenous vein grafts (SVG), which are used as grafts for coronary artery bypass grafting (CABG), are reported to have an inferior late patency rate, compared to arterial grafts such as grafts of the internal thoracic artery, and the use of artery conduits is reported to be increasing (Hamby R. I. et al., Circulation 60: 901-9 (1979); Virmami T. et al., Cardiovasc. Clin. 18: 41-59 (1988); Acinapura A. J. et al., Eur. J. Cardiothorac. Surg. 3 (4):321-5 (1989); Loop F. D. et al., N. Engl. J. Med. 314:1-6 (1986); Lytle B. W. et al., J. Throrac. Cardiovasc. Surg. 89:248-58 (1985); Grondin C. M. et al., Circulation 78 (Suppl I): I24-I29 (1989)). If these vein graft diseases (VGDs) can be prevented, the role of vein grafts would increase in view of their outstanding applicability.

In VGDs, stenosis of vein grafts caused by significant neointimal formation is observed when vein grafts are applied in arterial circulation. Cox et al. have investigated histological changes in vein grafts in arterial circulation. They revealed that fibrointimal proliferation associated with macrophage and neutrophil infiltration occurs within a year, and after one or more years it is atherosclerosis that becomes the major pathologic lesion (Cox J. L. et al., Prog. Cardiovasc. Dis. 34:45-68 (1991)). Angelini et al. also reported that three morphological processes contribute to medial and intimal thickening. The first process is rapid proliferation of smooth muscle cells in the media, occurring in the first week after transplant. The next process is the migration of smooth muscle cells, thickening, and synthesis of extracellular matrix in both the media and neointima, which occurs between one and four weeks after transplant. Finally, four weeks after transplant, a latter phase results, with slower proliferation of smooth muscle cells in the neointima (Angelini G. D. et al., J. Thorac. Cardiovasc. Surg. 103:1093-103 (1992)).

This kind of intimal hyperplasia development and progression mechanism is not fully understood, though physical damage to the vascular endothelium is thought to trigger abnormal proliferation of vascular smooth muscle cells (Nature 362: 801 (1993)). Damage in the vascular endothelium is thought to be the principal cause of arteriosclerotic intimal proliferation and restenosis. Specifically, changes in tension and shear-force caused by the application of vein grafts to the arterial circulation, as well as surgery itself, leads to a loss of the endothelium of vessel walls, and causes functional damage. Consequently, inflammatory cytokines and growth factors are activated, and medial smooth muscle cells differentiate from the medial layer, proliferating and migrating. Intimal hyperplasia are thought to be formed by their successive proliferation in the intimal layer (Bryan A. J. and Angelini G. D., Curr. Opin. Cardiol. 9:641-9 (1994); Angelini G. D. et al., J. Thorac. Cardiovasc. Surg. 99:433-9 (1990); Angelini G. D. et al., Ann. Thorac. Surg. 53: 871-4 (1992); Waters D. J. et al., Ann. Thorac. Surg. 56: 385-6 (1993); O'Neil G. S. et al., J. Thorac. Cardiovasc. Surg. 107:699-706 (1994); Schwartz L. B. et al., J. Vasc. Surg. 15:176-186 (1992); Galt S. W. et al., J. Vasc. Surg. 17: 563-70 (1993); Soyombo A. A. et al., Cardiovasc. Res. 27: 1961-7 (1993)).

Gene expression is controlled by transcription factors that bind to the transcriptional regulatory regions of genes. NFκB protein, which is known as a transcription factor, is a heterodimer protein comprising p65 and p50 subunits (Sen R. et al., Cell 46:705-16 (1986). NFκB is thought to function as a primary response switch when cells are externally stimulated. When NFκB is expressed in cytoplasm, it is activated by phosphorylation, migrates to the nucleus, and binds to a specific nucleotide sequence on the genomic DNA, called a “κB motif”, which comprises about ten nucleotides. It then activates the transcription of various genes. Genes known to be transcribed upon NFκB stimulation include: (1) cytokines such as interleukin−1, −2, −3, −6, −8, and −12, tumor necrosis factor-α (TNF-α), lymphotoxin-α and interferon-α, (2) receptors for granulocyte colony-stimulating factor, monocyte-macrophage colony-stimulating factor, granulocyte-monocyte/macrophage colony-stimulating factor, interleukin-2, and such, (3) stress proteins such as complement factor B, −C3, and −C4, and α1 acid glycoprotein, (4) leukocyte adhesion molecules such as ICAM-1, VCAM-1, E-selectin, (5) immunoregulatory molecules such as major histocompatibility complex class I and II molecules, T cell receptor α and β, and β2 microglobulin (Immunology Today 19: 80 (1998)).

NFκB binding protein (EP 584238) is conventionally known as a compound. that inhibits NFκB transcriptional activities. Aspirin and sodium salicylate, which are non-steroid type drugs, also inhibit NFκB activities at high concentrations (Kopp E. et al., Science 265: 956 (1994)). Furthermore, dexamethasone, a steroid-type drug, is reported to inhibit NFκB activation by inducing production of IκB, which is a regulatory subunit that binds to NFκB in the cytoplasm and keeps it in an inactive complex form (Scheinman R. I. et al., Science 270: 283 (1995); Auphan N. et al., Science 270: 286 (1995)).

On the other hand, a method employing a cis-element decoy is described in the specification of W095/11687, as one method for specifically obstructing the activation of gene transcription by specific transcription factors. Cis-element decoys are double-stranded DNA molecules with the activity of binding to specific transcription factors. By supplying cells with a large quantity of cis-element decoys, transcription factors bind to the cis-element decoys, rather than to target sequences on the genome, thus blocking the activation of gene transcription by transcription factors. Moreover, the present inventors demonstrated that various diseases caused by transcription factor NFκB, such as ischemic diseases, inflammatory diseases, autoimmune diseases, metastatic invasion of cancer, and cachexia can be treated and prevented using decoys against NFκB (W096/35430).

### Disclosure of the Invention

If intimal thickening is left untreated, it may induce cardiac angina, myocardial infarction, ischemic heart disease, aortic aneurysm, lower limb arteriosclerosis obliterans, and such. Thus it becomes a clinically important problem. Systemic drug therapies for preventing intimal thickening or restenosis, such as with antiplatelet agents, blood coagulation inhibitors, corticosteroids, and calcium channel blockers, have been examined. Intimal cell deficiency and platelet activation are closely involved in neointimal formation (Luscher T. F. et al., Curr. Opin. Cardiol. 8: 963-74 (1993)). Neointimal formation is known to be suppressed by the introduction of the eNOS gene (Von der Leyen H E. et al., Proc. Natl. Acad. Sci. USA 92: 113741 (1995)), anti-PDGF antibody (Ferns G. A. et al., Science 253: 1129-32 (1991), or anti-bFGF antibody (Olson N. E. et al., Am. J. Pathol. 140: 1017-23 (1992)). Further, the following are known as additional techniques for suppressing neointimal formation: introducing E2F decoys into damaged rat blood vessels using the HVJ-liposome method (Morishita R, et al., Proc. Natl. Acad. Sci. USA 92: 5855-9 (1995)), introducing the sdi-1 (p21) gene into rabbit jugular veins using the HVJ-liposome method, then autografting the vein onto the carotid artery (Bai HZ. et al., Ann. Thorac. Surg. 66 (3): 814-9 Sep; discussion 819-20 (1998)), introducing NFκB decoys into damaged rat blood vessels using the HVJ-liposome method (Yoshimura S. et al., Gene Ther. 8 (21) Nov: 1635-42 (2001)), and introducing E2F decoys into human vein grafts using a pressure-mediated method (Mann M. J. et al., Lancet 354: 1493-8 (1999)).

NFκB appears to be involved in the expression of neutrophil and macrophage chemotactic factors, adhesion molecules, and genes modulating cell cycle. In vein graft disease mechanisms, migration of macrophages and neutrophils, and the subsequent rapid-proliferation of smooth muscle cells in the media, occurs within one week after surgery. Then, between one and four weeks after vein grafting, extracellular matrix is synthesized in both the media and neointima. Therefore, the present inventors thought that by suppressing NFκB activation in the media within at least four weeks of the operation, excessive neointimal formation and subsequent enhanced atherosclerosis in SVGs used in CABG might also be suppressed. Thus, with the aim of reducing VGDs after CABG, the present inventors used model dogs to establish an experimental CABG model simulating actual CABG, and modified the pressure-mediated transfection method. To examine the effect of NFκB decoys on VGDs, the present inventors used this modified method in surgery to transfect NFκB decoys to a vein graft wall in CABG model dogs.

As a result, the effect of NFκB decoys in preventing VGDs, which was previously studied in vivo or using an alternative non-coronary artery bypass model, was proven in a large animal model. Thus, NFκB decoy transfection has been proven by histopathological methods to suppress not only differentiation and proliferation of medial smooth muscle cells, but also excessive production of extracellular matrix in the neointima. In fact, neointimal formation in the group transfected with NFκB decoys was significantly suppressed, compared with that of the group transfected with the scrambled decoy. Therefore, NFκB activation was suggested to induce differentiation and proliferation of medial smooth muscle cells in vein grafts, and transfection of NFκB decoys was indicated to effectively reduce neointimal formation.

The present inventors proved the four-week-long effect of preventing neointinal formation and the differentiation and proliferation of medial smooth muscle cells as a result of NFκB-decoy transfection into vein grafts. This shows the possibility of clinically applying NFκB decoys to reduce neointimal formation in vein grafts after CABG. The present invention provides the following methods and protective agents:

(1) A method for regulating transcription activated by the transcription factor NFκB in a part of a blood vessel or vascular graft, wherein the method comprises the step of contacting the graft with a decoy for the transcription factor NFκB.

(2) The method of (1), wherein the part of the vessel or vascular graft is a vein graft.

(3) The method of (1) or (2), wherein the method comprises contacting the NFκB decoy with a part of the vessel or vascular graft in vivo or ex vivo.

(4) The method of any one of (1) to (3), wherein the method comprises introducing the NFκB decoy into the vessel or vascular graft using a pressure-mediated method.

(5) The method of any one of (1) to (4), wherein the method suppresses neointimal formation in the graft by contact with the decoy against the transcription factor NFκB.

(6) An agent for protection from intimal thickening in a vascular graft, wherein the agent comprises an NFκB decoy.

(7) The agent for protection of (6), wherein the vascular graft is a vein graft.

(8) The agent for protection of (6) or (7), wherein the agent is for introducing an NFκB decoy into a vessel or vascular graft using a pressure-mediated method.

The NFκB decoys used in the present invention are not limited, as long as they are compounds with antagonistic activity specific to an NFκB binding site on a chromosome. For example, such compounds can be nucleic acids or their analogs. Oligonucleotides can be either DNAs or RNAs, and include not only naturally occurring nucleic acids, but also modified nucleic acids and pseudo-nucleotides. Moreover, these oligonucleotides can be either single-stranded or double-stranded, and can be either linear or circular. These NFκB decoys have either a sequence or structure that resembles a binding site (endogenous sequence) recognized by NFκB. That is, the nucleotide sequence of such a decoy comprises sequences homologous enough to be recognized and bound by NFκB.

An example of an endogenous sequence recognized by NFκB is GGGATTTCCC (SEQ ID NO: 1). Decoy sequences of the present invention include sequences that bind to the above-mentioned endogenous sequences under stringent conditions. These decoy sequences are preferably 50% or more, more preferably 70% or more, and even more preferably 90% or more identical to an endogenous sequence. Moreover, mutants of the above-mentioned sequences are included in these compounds. “Mutants” mean nucleic acids that comprise the above-mentioned sequences partially changed by deletion, substitution, addition, and/or insertion, and that specifically antagonize a nucleotide binding site that binds NFκB. On the other hand, folding, loop structures, twisting, crossing, helix structures, and such in nucleic acid decoys can mimic the structural characteristics of nucleotide sequences recognized by proteins, and these structures can be stabilized by non-nucleic acid components, such as cross-linking agents, as necessary.

More specifically, the NFκB decoys of the present invention include an endogenous binding sequence GGGATTTCCC (SEQ ID NO: 1), oligonucleotides comprising its complementary sequence (for example, 5′-CCTTGAAGGGATTTCCCTCC-3′ (SEQ ID NO: 2) used in the Examples), or such. It is known that the binding affinity of nucleic-acid binding proteins that recognize a specific nucleotide sequence can be increased by the nucleotide sequences of regions near the binding site. Therefore, sequences that promote NFκB binding can be placed for the above- described endogenous sequences or their analogs, as necessary. NFκB decoys include double- stranded oligonucleotides comprising one or several of the above-described sequences. Oligonucleotides that finction as NFκB decoys of the present invention include: oligonucleotides comprising thiophosphodiester bonds (S-oligo), in which oxygen atoms of the phosphodiester bonds have been replaced by sulfur atoms in order to suppress degradation in the body; and oligonucleotides in which phosphodiester bonds have been replaced by uncharged methylphosphates.

The NFκB decoys used in the present invention can be produced by chemical or biochemical synthesis methods used in the usual production of oligonucleotide compounds. For example, NFκB comprising nucleic acids can be produced by genetic engineering techniques, such as methods using a DNA synthesizer. Moreover, these nucleic acids can be amplified by PCR methods using synthesized DNA as a template as necessary, and also by inserting DNAs into appropriate cloning vectors. Furthermore, desired nucleic acids can be produced by digesting obtained nucleic acids with restriction enzymes and such, or by connecting them using DNA ligase and such. To stabilize these oligonucleotides in cells, the bases, sugars, and phosphate moieties of the nucleic acids can be chemically modified (alkylation, acylation, and such).

While protective agents comprising NFκB decoys of the present invention may comprise NFkB decoys alone, these agents can also be formulated to comprise at least one kind of additive and/or auxiliary, as required. Herein, examples of additives and auxiliaries include compounds such as lipids, cationic lipids, polymers, nucleic acid aptamers, peptides, and proteins that can enhance the migration of nucleic acids into cells, specifically transport compositions to particular cells, suppress the degradation of nucleic acids in cells, enhance the migration of nucleic acids into nuclei in cells, or stabilize nucleic acids during storage.

The protective agents comprising NFκB decoys of the present invention are not limited in their form, as long as they can be incorporated into cells or tissues in affected sites, and can be locally, parenterally, topically, or orally administered alone or mixed with appropriate carriers. For example, the agents can be in the form of liquids such as solutions, suspensions, syrups, liposome preparations (Szoka F. et al., Biochim, Biophys. Acta 601: 559 (1980) (reverse phase evaporation); Deamer D. W. et al., Ann. N. Y. Acad. Sci. 308:250 (1978) (ether injection); Brunner J. et al., Biochirn. Biophys Acta 455: 322 (1976) (surfactant method)), and emulsions, or solids such as tablets, granules, powders, capsules. In these drug formulations, additives such as various carriers, auxiliaries, stabilizers, and lubricants can be added as necessary. Protective agents comprising NFκB decoys of the present invention are preferably administered to patients by a pressure-mediated transfection method. For example, NFκB decoys can be introduced to a patient's vessels or vascular grafts by immersion in physiological saline, and using the pressure-mediated transfection method at 10-500 mg for 5 to 30 minutes.

Vessels or vascular grafts to be contacted with NFκB decoys of the present invention include various vessels, such as the internal thoracic artery and the great saphenous vein. In particular, the effect of the protective agents comprising NFκB decoys of the present invention in vein-derived grafts is expected to be high, and therefore such vein-derived grafts are particularly preferred as vascular grafts to be targeted in the present invention.

Pharmaceutical agents that comprise an NFκB decoy of the present invention as a major component comprise a sufficient amount of NFκB decoy to prevent intimal formation in vessels or vascular grafts. Although the dose of a NFκB decoy varies depending on conditions such as patient age, condition and weight, the type of decoy used, and the administration form, one skilled in the art can select suitable doses in view of these conditions. If the pressure-mediated transfection method is used to administer a protective agent comprising an NFκB decoy of the present invention, the agent can generally be immersed in physiological saline and such at a concentration of 10-500 μmol/l, and administered.

### BRIEF DESCRIPTION OF THE DRAWINGS

FIG. 1 is a schematic drawing of the pressure-mediated transfection method.

FIG. 2 is a photograph showing a dog CABG model, in which a saphenous vein graft was inserted between the descending aorta and the left anterior descending coronary artery.

FIG. 3 is a photograph showing the results of histopathological investigations. FIGS. 3-1-a and 3-1-b show the results of histopathological evaluation of the efficiency of the pressure-mediated transfection of FITC-ODNs, using FITC-labeled ODNs. FIGS. 3-2-a and 3-2-b show the results of histopathological evaluation of neointimal formation, using hematoxylin-eosin staining. FIGS. 3-3-a and 3-3-b show the results of evaluating medial smooth muscle cell proliferation, using α-actin staining. FIGS. 3-4-a and 3-4-b show the results of evaluating the excessive production of extracellular matrix in neointima, using Masson-trichrome staining.

FIG. 4 is a graph showing the neointimal area to medial area ratio.

FIG. 5 is a graph showing proliferating cell nuclear antigen (PCNA) index (%).

### BEST MODE FOR CARRYING OUT THE INVENTION

1) Decoy Preparation

Double-stranded oligonucleotides with the following sequences were used in the experiment:

NFκB decoy:

(SEQ ID NO: 2)

5′-CCTTGAAGGGATTTCCCTCC-3′
3′-GGAACTTCCCTAAAGGGAGG-5′
Scrambled decoy:

(SEQ ID NO: 3)

5′-TTGCCGTACCTGACTTAGCC-3′
3′-AACGGCATGGACTGAATCGG-5′

These decoys were stored at −20° C. until the day of surgery, and then kept at 4° C. until transfection. Decoys were prepared for transfection at room temperature in 0.9% physiological saline injection solution, at a concentration of 40 μmol/L.

2) Assessment of Conditions for Pressure-Mediated Transfection

Mann et al. reported detailed data concerning pressure-mediated transfection (Mann M. J. et al., Proc. Natl. Acad. Sci. USA 96: 6411-6 (1999)). Preliminary examinations of transfection efficiencies at various transfection pressures and times were conducted. These preliminary 25 experiments showed that transfection efficiency at 200 mmHg for 20 minutes was not much different from that reported by Mann et al., at 300 mmHg for ten minutes (data not shown). Rather, when pressure-mediated transfection during leg artery bypass grafting surgery was performed at 300 mmHg for ten minutes, neointimal formation in vein grafts was significant, even in groups transfected with NFκB decoys. Moreover, three out of six samples in a group transfected with a scrambled decoy showed complete occlusion. Therefore, the conditions of 300 mmHg for ten minutes were not considered optimal, at least for decoy transfection into vein grafts for coronary artery bypass grafting. Thus, in the experiments below, transfection was performed at 200 mmHg for 20 minutes.

3) The Canine CABG Model

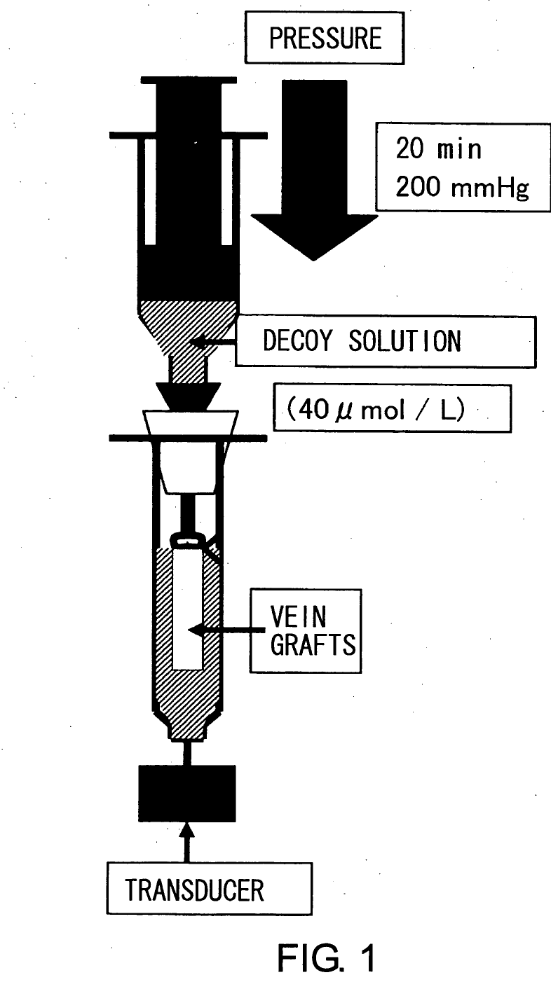

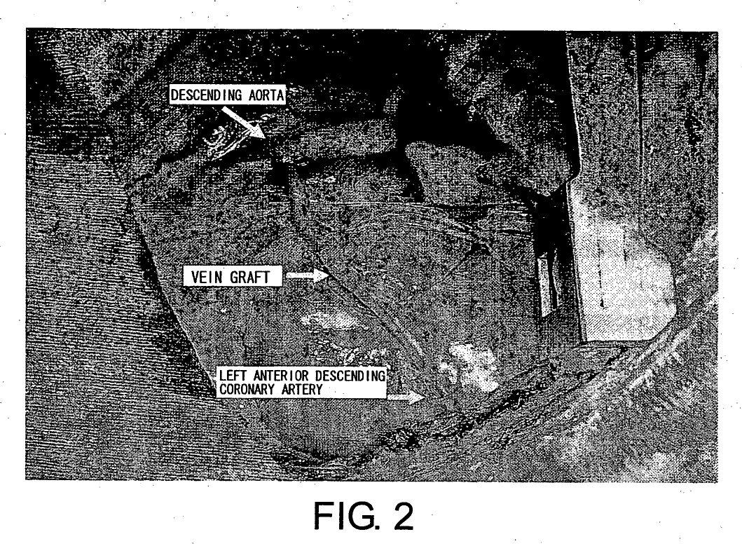

Mongrel dogs (NRB; Nihon Nosan, Kanagawa, Japan or HBD; Oriental Yeast Corporation, Osaka, Japan) weighing 18 to 20 kg and fed with a standard food were used. After anesthetizing the dogs with ketamine (5 mg/kg body weight, intramuscular injection), intratracheal intubation was performed. Saphenous vein grafts were collected from the left hind leg of the dogs, kept under general anesthesia by inhalation of 1.5% sevoflurane. To expose approximately 10 cm of the saphenous vein, the outer part of the legs was incised along the anteroposterior axis. The vein was cut from surrounding tissue using a “no touch technique” (Gottlob R., Minerca Chir. 32: 693-700 (1977)) and all side branches were ligated with 4-0 silk ligature. The vein was then recovered from the dog and washed with heparinized 0.9% saline solution without distension (Angelini G. D. et al., Cardiovasc. Res. 21: 902-7 (1987)). The above veins were then stored in the same solution at room temperature for about 60 minutes. A left fourth intercostal thoracotomy was performed, and a scrambled decoy (SD groups; n=5) or NFκB decoy (ND groups; n=5) solution (40 μmol/L) was introduced into the vein graft wall by the method of Mann et al. (the pressure-mediated transfection; Proc. Natl. Acad. Sci. USA 96: 6411-6 (1999)) at 2000 mmHg for 20 minutes (FIG. 1). After intravenous injection of heparin (100 U/kg body weight), the saphenous vein graft was interposed between the descending aorta (descending Ao.) and the left anterior descending coronary artery (LAD) without cardiopulmonary bypass and cardiac arrest (i.e., beating heart surgery). That is, an end-to-side anastomosis between the vein graft and the left anterior descending coronary artery was performed on the beating heart with 7-0 Prolene suture (Ehicon, Inc., USA) using an “Octopus” (Medotronic Inc., USA) stabilizer, and the other end of the vein graft was sutured to the descending aorta with 6-0 Prolene, in an end-to-side fashion. The LAD proximal region was sewn with 4-0 Prolene (FIG. 2).

An antibiotic (CEZ, Fujisawa pharmaceuticals, Japan) was administered to the dogs three days after the operation. The dogs were sacrificed four weeks after operating, and the grafts were carefully recovered. The experiments were carried out in accordance with guidelines approved by the Animal Experiment Committee of Osaka University Medical School. All animals were treated in compliance with the “Principles of Laboratory Animal Care” established by the National Society for Medical Research and the “Guide for the Care and Use of Laboratory Animals”, published by the United States National Institutes of Health (NIH) (NIH publication No. 86-23, revised on 1985). The grafts were dissociated and briefly washed in 0.9% physiological saline solution. The middle portion of the grafts was then divided into three parts, which were approximately 5 mm thick. These pieces were frozen in an OCT compound (Miles Scientific, USA) in a freezing mold placed in liquid nitrogen.

4) The distribution of oligodeoxynucleotides (ODNs) introduced by pressure-mediated transfection

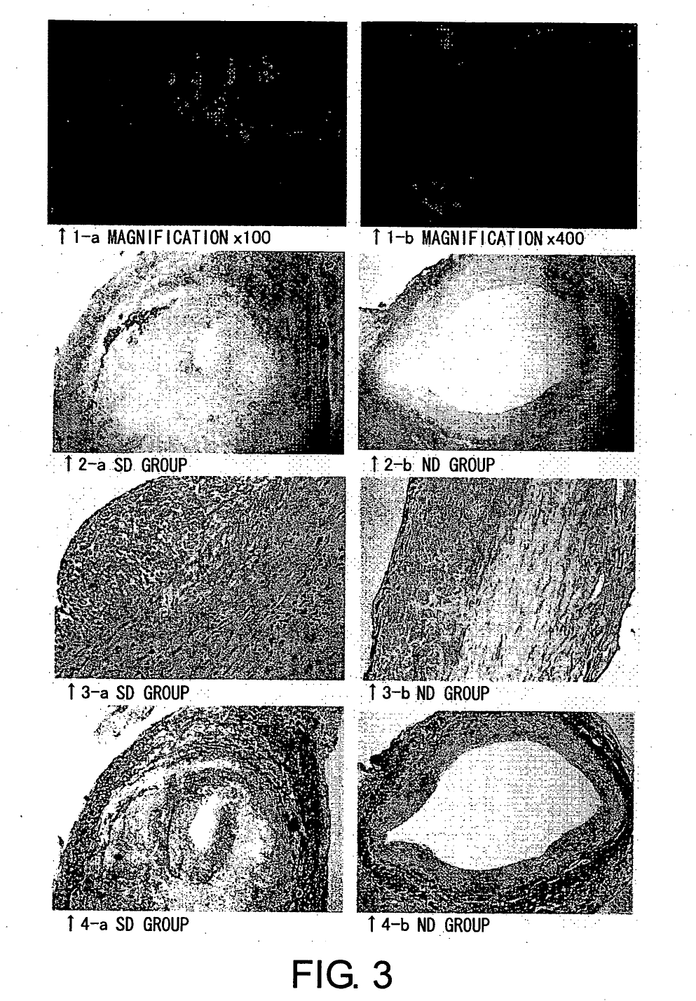

Fluorescent isothiocyanate (FITC)-labeled ODNs (FITC-ODNs) were used to histopathologically evaluate the distribution of oligodeoxynucleotides (ODNs) by pressure-mediated transfection in transverse sections (about 5 μm thickness) of a fresh-frozen block of each graft (FIGS. 3-1-a, and 3-1-b). After evaluating FITC-ODN distribution resulting from pressure-mediated transfection, the same transverse sections were stained with hematoxylin-eosin (HE). The decoy. transfection efficiencies were then calculated. FITC-positive and hematoxylin-positive nuclei were counted using “NIH image” under x200 magnification. The average decoy transfection efficiency (defined as the ratio of FITC-positive nuclei to total nuclei) was 77±20%.

5) Measurements of neointimal and medial areas

Transverse sections (about 5 μm thick) of fresh-frozen blocks of each graft were stained with HE. These HE-stained transverse sections were used to measure the areas of the neointima and media, and the ratio of neointimal area to medial area, using computerized image analysis software “NIH image”. Neointimal thickening of the ND group was significantly suppressed compared to that of the SD group (FIG. 3-2-a, and 3-2-b). Table 1 shows the average neointimal and medial areas in the transverse sections, measured four weeks postoperative.

TABLE 1

| | SD (n = 5) | ND (n = 5) |
|---|---|---|
| Neointimal area (mm²) | 2.63 ± 1.00 | 0.88 ± 0.66* |
| Medial area (mm²) | 1.86 ± 0.82 | 1.41 ± 0.55* |

The average neointimal and medial areas in the transverse sections, measured four weeks after the operation, are shown. The average neointimal area of the ND group was significantly suppressed compared to that of the SD group (*p<0.05 against SD group).

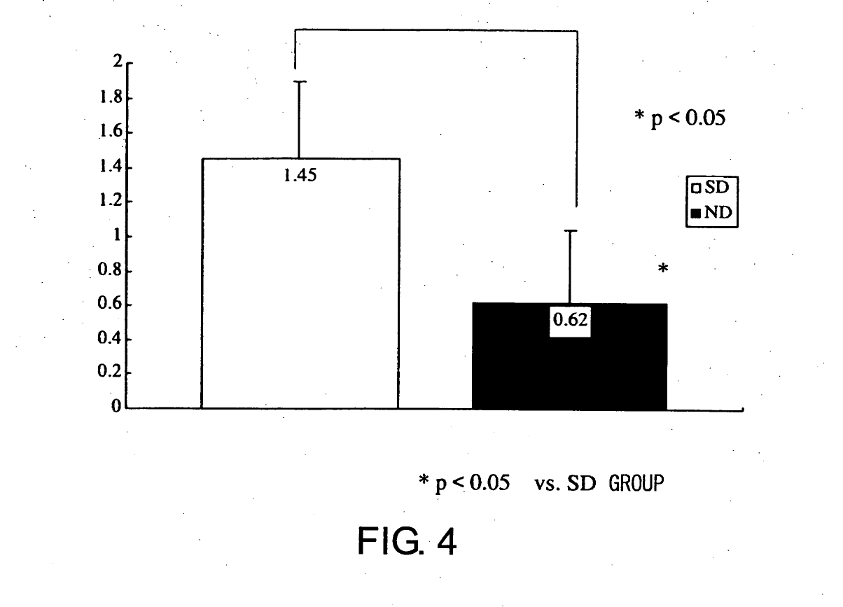

Furthermore, the ratio of neointimal area to medial area is shown in FIG. 4. The neointimal to medial area ratio (neointimal area (mm²)/medial area (mm2)) of the ND group was 0.62±0.43, which was significantly lower than the SD group's 1.45±0.45 (*p<0.05). All values are indicated as “means±SD”. A Student's unpaired t-test was used to compare the two groups. Statistical significance was taken as P<0.05.

6) Immunohistochemical evaluation

6-1. Proliferation of medial smooth muscle cells

Proliferation of medial smooth muscle cells was immunohistochemically evaluated using monoclonal antibodies against smooth muscle-specific α-actin. Frozen sections (about 5 μm thick) were prepared from a fresh-frozen tissue block, and monoclonal antibody against α-actin specific to α-actin smooth muscle (Histofine, Nichirei, Japan) was used. Immunohistochemical staining was performed using the immunoperoxidase, avidin-biotin complex system with nickel chloride color, and by a modified method of Bai et al. (Arterioscler. Thromb. 14: 1846-53 (1994)). The staining result was measured using computerized image analysis software “NIH image”. The α-actin staining revealed a trend to inhibition of medial smooth muscle cell proliferation by transfection of the NFκB decoys (FIGS. 3-3-a and 3-3-b).

6-2. Differentiation and proliferation of medial smooth muscle cells

Proliferating cell nuclear antigen (PCNA) staining was performed according to the same protocol used for α-actin staining in 5-1 above, except that anti-PCNA monoclonal antibody (PC-10, DAKO) was used as a specific marker for smooth muscle cells and differentiating and proliferating cells. PCNA-positive and hematoxylin-positive nuclei in the media were counted using “NIH image” under x 200 magnification. Cell proliferation frequency was expressed as a PCNA index defined as the ratio of PCNA-positive nuclei to total nuclei.in the media.

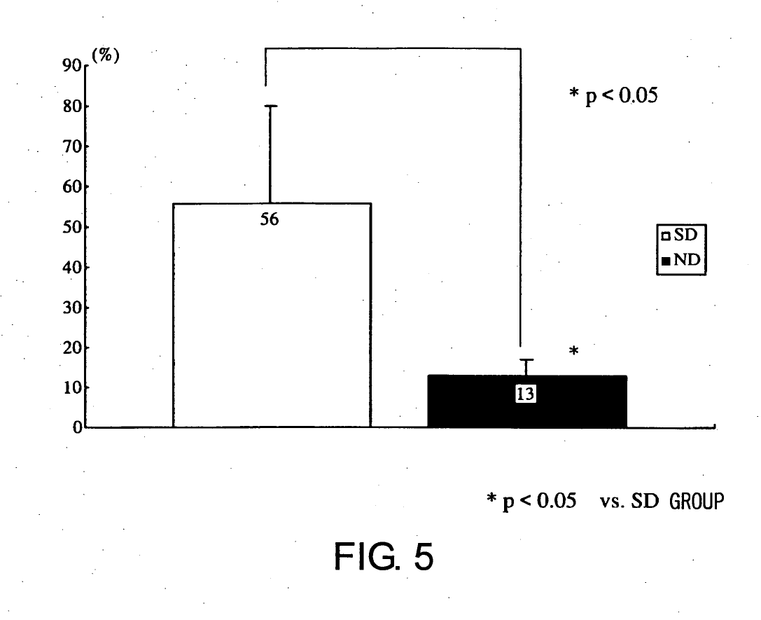

A monoclonal antibody against PCNA was used to evaluate the differentiation and proliferation of medial smooth muscle cells in the four-week postoperative cross-sections. The PCNA index of the ND group was 13±4*%, which was lower than the 56±24% of the SD group (*p<0.05 against the SD group) (FIG. 5). All values are expressed as “means±SD”. A Student's unpaired t-test was used to compare the two groups. Statistical significance was taken as p<0.05.

6-3. Extracellular matrix staining

Masson-trichrome staining was performed as the extracellular matrix staining. Suppression of excessive extracellular matrix production in the neointima of the ND group was observed using Masson-trichrome staining (FIGS. 3-4-a, and 3-4-b).

Industrial Applicability

The VGD preventive effect of NFκB decoys, which has previously been investigated in vivo and using alternative non-coronary bypass models, has been demonstrated in a large animal model. The present invention uses histopathological methods to prove that transfection of NFκB decoys suppresses not only medial smooth muscle cell differentiation and proliferation, but also the production of excessive extracellular matrix in neointima. The present invention has demonstrated the effect of transfecting NFκB decoys into vein graft walls on preventing neointimal formation and medial smooth muscle cell differentiation and proliferation, and has shown that NFκB decoys can be clinically applied to reduce neointimal formation in vein grafts after CABG.

1. A method for regulating transcription activated by the transcription factor NFκB in a part of a blood vessel or vascular graft, wherein the method comprises the step of contacting the graft with a decoy for the transcription factor NFκB.

2. The method of claim 1, wherein the part of the vessel or vascular graft is a vein graft.

3. The method of claim 1, wherein the method comprises contacting the NFκB decoy with a part of the vessel or vascular graft in vivo or ex vivo.

4. The method of claim 1 wherein the method comprises introducing the NFκB decoy into the vessel or vascular graft using a pressure-mediated method.

5. The method of claim 1 wherein the method suppresses neointimal formation in the graft by contact with the decoy against the transcription factor NFκB.

6. An agent for protection from intimal thickening in a vascular graft, wherein the agent comprises an NFκB decoy.

7. The agent for protection of claim 6, wherein the vascular graft is a vein graft.

8. The agent for protection of claim, wherein the agent is for introducing an NFκB decoy into a vessel or vascular graft using a pressure-mediated method.
